(19) United States
(12) Patent Application Publication
(10) Pub. No.: US 2005/0172964 A1
(43) Pub. Date: Aug. 11, 2005

(54) Medicament dispenser

(76) Inventors: Gregor John McLennan Anderson, Ware Hertfordshire (GB); Philip William Farr, Hertfordshire (GB); Andrew Michael Kelly, Hertfordshire (GB)

Correspondence Address: DAVID J LEVY, CORPORATE INTELLECTUAL PROPERTY;GLAXOSMITHKLINE, FIVE MOORE DR., PO BOX 13398, RESEARCH TRIANGLE PARK, NC, 27709-3398, US

(21) Appl. No.: 10/512,667

(22) PCT Filed: Apr. 24, 2003
(86) PCT No.: PCT/EP03/04403

(30) Foreign Application Priority Data
Apr. 26, 2002 (GB) 0209526.3

(51) Int. Cl. A61M 15/00; A61M 16/00

(52) U.S. Cl. 128/203.21

(57) ABSTRACT

There is provided a medicament dispenser for use with a medicament carrier having a plurality of pockets for containing medicament wherein said pockets are spaced along the length of and defined between two peelable sheets secured to each other. The dispenser has an opening mechanism for opening received pockets of the medicament carrier. The mechanism includes an indexer for indexing pockets of a medicament carrier in use with said medicament dispenser. The indexer comprises an index ratchet, which is moveable between a locked position in which said ratchet locks a lid driver and a release position in which the ratchet releases the lid driver. Actuation of the medicament dispenser releases the index ratchet from the lid driver to enable drivable peeling of the lid sheet from the base sheet of the pocket at an opening station.

### TECHNICAL FIELD

The present invention relates to a medicament dispenser for dispensing medicament. The invention particularly relates to a device for use in dispensing medicament in powder or tablet form.

### BACKGROUND TO THE INVENTION

The use of inhalation devices in the administration of medicaments, for example in bronchodilation therapy is well known. Such devices generally comprise a body or housing within which a medicament carrier is located. Known inhalation devices include those in which the medicament carrier is a blister strip containing a number of discrete doses of powdered medicament. Such devices usually contain a mechanism of accessing these doses, usually comprising either piercing means or means to peel a lid sheet away from a base sheet. The powdered medicament can then be accessed and inhaled. Such a mechanism may also be used for dispensing medicament in tablet form wherein peeling away the lid sheet from the base sheet reveals a tablet for removal and subsequent consumption.

It is an object of the present invention to simplify the internal mechanism of a medicament dispenser for dispensing medicament in powder or solid form from a medicament carrier as described supra.

It is a further object of the present invention to provide a medicament dispenser whose operation is straightforward and non-complex and in particular to minimise the number of separate steps involved in preparing the device for use. This is especially relevant where the device is designed for use in the delivery of medicament in emergency or rescue situations (e.g. asthma attacks) where simplicity and ease of use is paramount.

It is a further object of the invention to provide a medicament dispenser device suitable for use with a large number of discrete doses but which is of an acceptable size for use by patients.

### SUMMARY OF THE INVENTION

According to one aspect of the present invention there is provided a medicament dispenser for use with a medicament carrier having a plurality of pockets for containing medicament wherein said pockets are spaced along the length of and defined between two peelable sheets secured to each other, said dispenser having an internal mechanism for accessing said medicament contained within said medicament carrier, said mechanism comprising,

a) an opening station for receiving a pocket of said medicament carrier;

b) associated with said opening station, a peeler for peelably opening a pocket, said peeler including a lid driver for drivably peeling a lid sheet from a base sheet of said pocket;

c) an outlet, positioned to be in communication with an opened pocket through which a user can access medicament from such an opened pocket; and

d) an indexer for indexing pockets of a medicament carrier in use with said medicament dispenser,

wherein said indexer comprises an index ratchet which is moveable between a locked position in which said ratchet locks said lid driver and a release position in which the ratchet releases the lid driver, and actuation of said medicament dispenser releases said index ratchet from the lid driver to enable drivable peeling of said lid sheet from said base sheet of the pocket at the opening station.

The medicament dispenser herein is suitable for dispensing medicament from a medicament carrier. The term medicament carrier herein is used to define any suitable form of carrier. Suitably, the medicament carrier is in the form of an elongate strip or tape.

The medicament carrier has a plurality of pockets for containing medicament wherein said pockets are spaced along the length of and defined between two peelable sheets secured to each other. Embodiments are envisaged in which the medicament carrier has from 5 to 200 pockets, particularly from 10 to 60 pockets.

In one aspect, the medicament carrier comprises a blister pack in laminate form. Suitably, the laminate comprises material selected from the group consisting of metal foil, organic polymeric material and paper. Suitable metal foils include aluminium or tin foil having a thickness of from 5 to 100 μm, preferably from 10 to 50 μm, such as 20 to 30 μm. Suitable organic polymeric materials include polyethylene, polypropylene, polyvinyl chloride and polyethylene terephthalate.

Access to the medicament dose portions comprised within the pockets of the elongate strip form carrier is by any suitable access means including tearing, piercing or peeling apart the relevant pockets.

One suitable blister pack form medicament carrier comprises a peelable blister strip. Suitably, the peelable blister strip comprises a base sheet in which blisters are formed to define pockets therein for containing distinct medicament dose portions and a lid sheet which is hermetically sealed to the base sheet except in the region of the blisters in such a manner that the lid sheet and the base sheet can be peeled apart. The base and lid sheets are typically sealed to one another over their whole width except for the forward end portions where they are typically not sealed to one another at all. Thus, separate base and lid sheet forward end portions are presented at the end of the strip. The respective base and lid sheets are peelably separable from each other to (e.g. separately) release the contents of each pocket.

Suitably, the lid sheet comprises at least the following successive layers: (a) paper; adhesively bonded to (b) polyester; adhesively bonded to (c) aluminium foil; that is coated with a heat seal lacquer for bonding to the base sheet. The thickness of each layer may be selected according to the desired properties but is typically of the order of from 5 to 200 micron, particularly from 10 to 50 micron.

Suitably, the base sheet comprises at least the following successive layers: (a) oriented polyamide (OPA); adhesively bonded to (b) aluminium foil; adhesively bonded to (c) a third layer comprising a polymeric material (e.g. polyvinyl chloride).

Various known techniques can be employed to join the lid and base sheet and hence to seal the blisters of the peelable blister strip. Such methods include adhesive bonding, hot metal bonding, hot metal welding, radio frequency welding, laser welding, ultrasonic welding and hot bar sealing. The lid sheet and base sheet of the peelable blister strip are particularly sealable by ‘cold form’ sealing methods, which are conducted at lower temperatures than conventional heat sealing methods. Such ‘cold form’ sealing methods are of particular utility where the medicament or medicament formulation for containment within the blister is heat sensitive (e.g. degrades or denatures on heating). Suitable ‘cold form’ sealing methods are conducted at a temperature in the range of 150-250° C., more preferably, 210-240° C.

In one aspect, the blister pockets are provided to each carrier in uniform series. In particular, the spacing (i.e. pitch) between each pocket is uniform throughout the series. In other aspects however, the spacing (i.e. pitch) may vary throughout the series (i.e. be non-uniform). In specific examples, the pitch may progressively decrease or progressively increase throughout the series. Corresponding variation of ratchet spacing of the index ratchet is also therefore envisaged.

The medicament dispenser has an internal mechanism for accessing medicament carried by said medicament carrier. The mechanism comprises a receiving station for receiving a pocket of the medicament carrier.

The mechanism further comprises a peeler for peeling apart a pocket of the medicament carrier. In one aspect herein, each blister pocket is peeled apart about a defined beak, wedge or roller form feature of the dispenser.

An outlet is positioned to be in communication with the distinct medicament dose portions releasable by said a release to enable their dispensing to the patient. The outlet may have any suitable form. In one aspect, it has the form of a mouthpiece. In another aspect, it has the form of a nozzle for insertion into the nasal cavity of a patient.

The internal mechanism includes an indexer. In use, it may be appreciated that the indexer prevents further movement of the medicament carrier until the required dose of medicament has been dispensed.

Suitably, when the medicament dispenser is in use, the lid driver applies tension to the lid sheet. Also suitably, the base sheet is untensioned in use.

In one use aspect, the index ratchet is initially positioned (‘locked position’) such as to prevent movement of the lid driver. In response to actuation of the dispenser however, the index ratchet reversibly released (‘release position’) to enable defined movement of the lid driver. The defined movement (e.g. distance of travel) corresponds generally to that required to peel the lid sheet from the base sheet of one pocket thereby opening the pocket for release of a dose (generally, one pocket's worth) of medicament. Once the defined movement has been completed, the index ratchet again locks (‘locked position’) and thereby acts such as to prevent further movement of the lid driver and hence further drivable peeling action, until after the released dose of medicament has been dispensed to the patient.

The index ratchet may have any suitable form. In aspects, it comprises a ratchet arm that is suitably, mounted (e.g. pivotally or rotationally) to the dispenser. The ratchet arm is generally shaped (e.g. with a dog-leg end) to engage a ratchet index (e.g. a toothed rack) that may in aspects, have linear or circularly arranged ratchet elements (e.g. teeth). The engagement between arm and rack occurs in ratcheted fashion.

Suitably, the medicament dispenser further comprises a back ratchet to prevent reverse movement of the internal mechanism and/or medicament carrier therein. In particular, the back ratchet prevents reverse movement of the index ratchet.

Suitably, the medicament dispenser further comprises an indexing lever (e.g. coupled to the indexer) for actuating said dispenser (e.g. to move the index ratchet from the locked to release position, thereby allowing drivable peeling of the lid sheet by the lid driver).

Suitably, the medicament dispenser further comprises an end-locking mechanism to lock the indexer and/or lid driver after opening of the final pocket of a medicament carrier. In one aspect, the end-locking mechanism results in a breakage of a key part of the internal mechanism thereby preventing further use.

In aspects, the base sheet (once detached from the lid sheet) is freely movable within the device. Embodiments are envisaged in which the lid driver—acting on the lid sheet—draws the medicament carrier through the internal accessing mechanism.

In one aspect, the lid driver comprises a rotary drive mechanism. In another aspect, the lid driver comprises a linear drive mechanism.

Suitably, the medicament dispenser additionally comprises an actuation or dose counter, wherein actuation of said counter is coupled to the actuation of the medicament dispenser.

The counter may count the number of doses left to be taken or the number of doses taken. In one aspect, the counter is electronic. Alternatively, the counter is mechanical.

In one aspect, actuation of the counter is coupled to the release of the index ratchet.

Suitably, the medicament dispenser additionally comprises an indexing lever for actuating said dispenser.

In one aspect, the indexing lever comprises cam means for moving the index ratchet between locked and release positions.

Suitably, the medicament dispenser additionally comprises a moveable cover, wherein movement of said cover results in release of the index ratchet.

Suitably, the lid driver comprises a mangle. The lid sheet passes through two rotating wheels, which act as a mangle and is gripped at the point of contact with the wheels. The used portion of the lid sheet is collected in a chamber after it has passed through the mangle.

In another aspect, the lid driver comprises a roller. Suitably, said roller is composed of a polymeric rubber and is positioned next to a guide wall. Suitably, said roller has a smooth surface. Alternatively said roller has a knurled surface. The roller grips the lid sheet as it passes from the point at which it is separated from the base sheet through the space between the roller and the guide wall and the used portion of the lid sheet is then collected in a chamber. The roller has the advantage over the mangle described above in that a greater degree of contact between the roller wheel and the lid sheet occurs—the lid sheet is squeezed through the roller and may pass around about ⅓ of the roller wheel. This provides a higher level of grip and pulling force than with a mangle. The force required to turn the roller is constant throughout the use of the device and does not vary according to how much of the lid sheet has been peeled away from the base sheet. The roller also has the advantage that the lid sheet does not have to be looped around or fixed to the roller before use of the device, therefore simplifying assembly of the device and reducing costs.

In one aspect, the lid driver and/or the index ratchet are operated by an electronic drive system. The electronic drive system may also be used in conjunction with a mechanical drive system. The electronic drive system may include a DC motor.

The electronic drive means typically comprises a motor, preferably an electrically powered motor. The motor may provide linear or rotary drive, but in general, rotary motors are most suitable. The motor may for example, comprise a DC electric motor, a piezoelectric (PZ) motor, an ultrasonic motor, a solenoid motor or a linear motor. Preferably, the electronic drive system comprises a DC motor, a PZ motor or an ultrasonic motor.

The use of ultrasonic motors is particularly preferred since they offer advantages over conventional motors in terms of weight, size, noise, cost and torque generated. Ultrasonic motors are well known in the art and are commercially available (e.g. BMSTU Technological Cooperation Centre Ltd, Moscow, Russia; Shinsei Corporation, Tokyo, Japan).

Ultrasonic motors do not use coils or magnets but comprise a piezo-electric ceramic stator which drives a coupled rotor. The stator generates ultrasonic vibrations which in turn causes rotation of the rotor. While regular DC motors are characterised by high speed and low torque, requiring reduction gearing to increase torque, ultrasonic motors attain low speed and high torque, thus eliminating the need for reduction gearing. Furthermore, these motors are lightweight and compact, lacking coils and magnets, and are noiseless as the ultrasonic frequencies used are not audible to the human ear.

Suitably, the dispenser further comprises actuating means for actuating said electronic drive system. Said actuating means may take the form of a switch, push-button, or lever.

Suitably, the internal mechanism additionally comprises a first chamber in which the strip is initially housed and from which it is dispensed and a second chamber to receive the used portion of the base sheet after it has been indexed and separated from the lid sheet.

Suitably, a wall separates said first chamber and said second chamber.

Suitably, said wall is movable to adjust the size of said first and second chambers.

Suitably, the wall is pivotally mountable. Alternatively, the wall is slidably mountable.

Suitably, the wall is flexible to allow changes in the relative size of said first and second chambers.

Suitably, the internal mechanism further comprises a third chamber to receive the used portion of the lid sheet and a fourth chamber that houses the index ratchet. The fourth chamber may communicate via a slit, which in turn extends upwardly within a mouthpiece and communicates with air inlets.

In one aspect, the internal mechanism for accessing said medicament contained within said medicament carrier is housed within a cassette.

According to another aspect herein there is provided a medicament dispenser for dispensing medicament comprising: a body; a holder, shaped to fit within said body and movable relative to said body; and receivable by said holder, a cassette containing said medicament carrier.

Suitably, movement of the holder relative to the body results in movement of the cassette between a first position and a second position such that the cassette is reversibly removable from the holder when the cassette is in the second position.

Suitably, the first position comprises a dispensing position. Preferably the second position comprises a non-dispensing position. The cassette is therefore only removable from the holder when the cassette is in the non-dispensing position.

Suitably, the holder and body include attaching means to attach the holder to the body. Preferably, said attaching means comprise a snap fit mechanism. Preferably, said snap fit mechanism comprises a pin and hole system.

Suitably, the holder is pivotally movable relative to the body.

Alternatively, the holder is rotationally movable relative to the body.

Suitably, the holder additionally comprises a stop to limit movement of the holder relative to the body. The stop abuts against the edge of the body at two points when it is rotated. At these points the holder may be designed to click into place. Therefore when the stop abuts one body edge then it is clicked into the dispensing position and when the stop abuts the other body edge then it is clicked into the non-dispensing position.

Alternatively, the holder is slidably movable relative to the body.

Suitably, the holder additionally comprises a catch to retain the cassette. The catch may for example comprise a sprung pin that fits into a hole or an integral catch that deforms when pressed allowing removal of the cassette.

Suitably, the catch is child resistant. Child resistance may be realised by having a system that forces the user to perform two actions at once to remove the cassette. Other features of the catch may include shock or impact resistance, the ability to lock the catch and orientation features to ensure that the cassette can only be inserted one way. The catch should also be easy to manufacture and assemble, be robust, be composed of a minimal number of components and intrude minimally into the space into which the cassette is inserted.

Suitably, the holder includes guide means to guide the cassette into the holder. Preferably said guide means comprise guide rails. Alternatively the guide means comprise grooves, indentations or other shaping or surface details to define a ‘lock and key’ relationship between the holder and the cassette. Colour guides, arrows and any other surface markings may also be employed.

Suitably, the cassette additionally comprises an indexing lever. The indexing lever has a finger tab located outside the body of the cassette. The rest of the indexing lever is located within the cassette. The indexing lever may have teeth at its tail end and/or teeth along its mid portion.

Suitably, the cassette additionally comprises a mouthpiece.

The medicament dispenser may also be designed for nasal inhalation of a powdered medicament and may therefore incorporate a nosepiece as an alternative to a mouthpiece. If the medicament is in solid form, the dispenser may incorporate an exit channel for tablet release.

Suitably, the body covers the mouthpiece and/or the indexing lever when the cassette is in the non-dispensing position. This avoids the need for a separate cover and protects the mouthpiece from the ingress of dirt and contaminants during storage.

Suitably, the cassette additionally comprises a raised portion to fit against the holder. The raised portion is located at the opposite end of the cassette to the mouthpiece/nosepiece/exit and indexing lever and prevents the incorrect insertion of the cassette into the holder since it is too wide to fit into the holder. The raised portion is shaped such that it fits against a cut away part of the holder. Preferably, said raised portion includes a section that is raised to define a grip portion.

Suitably, at least a portion of the holder and body are shaped for ease of grip by the user.

Suitably, operation of the device may be performed with one hand.

The refillable device may be assembled as follows. The holder is snap fitted into the body. The cassette is assembled separately. The body of the cassette is formed, preferably in two sections with any necessary spindles or integral components formed into the base. Individual components such as the indexer, lid drive mechanism, guide portions etc are then assembled into the base. Finally the medicament containing blister strip (or other suitable medicament carrier) may be inserted into the cassette. This may be wound into the device before the lid is attached to the cassette and the cassette sealed. Alternatively, the cassette may be formed completely apart from a hole left in its side for insertion of the blister strip or medicament carrier. The hole may then be sealed to complete the cassette. This second method of inserting the medicament carrier into the device has the advantage that it is much simpler.

Suitably, the medicament dispenser additionally comprises an electronic data management system. The electronic data management system has input/output capability and comprises a memory for storage of data; a microprocessor for performing operations on said data; and a transmitter for transmitting a signal relating to the data or the outcome of an operation on the data.

Suitably, the electronic data management system is arranged to be responsive to or activated by the voice of a user. Thus, for example the system may be switched on or off in response to a voice command.

The electronic data management system may be integral with the body. Alternatively, the electronic data management system forms part of a base unit that is reversibly associable with the body.

Suitably, the medicament dispenser additionally comprises a data input system for user input of data to the electronic data management system. Preferably, the data input system comprises a man machine interface (MMI) preferably selected from a keypad, voice recognition interface, graphical user interface (GUI) or biometrics interface.

Energy may be conserved by a variety of means to enable the device to operate for longer on a given source of energy, such as a battery. Energy conservation or saving methods have additional advantages in terms of reducing the size requirements of the power source (e.g. battery) and thus the weight and portability of the medicament dispenser.

A variety of energy saving methods is available which generally involve reducing power consumption. One such method is to use a clock or timer circuit to switch the power on and off at regular or predetermined intervals. In another method the system can selectively switch on/off specific electronic devices, such as visual display units or sensors, in order to power these devices only when they are required to perform a particular sequence of events. Thus different electronic devices may be switched on and off at varying intervals and for varying periods under control of the system. The power sequencing system may also respond to a sensor, such as a motion or breath sensor, which is activated on use of the device.

Low power or “micropower” components should be used within the electronics where possible and if a high power device is required for a particular function this should be put into a low power standby mode or switched off when not required. Similar considerations apply in the selection of transducers. Operation at low voltage is desirable since power dissipation generally increases with voltage.

For low power digital applications complementary metal oxide semi-conductor (CMOS) devices are generally preferred and these may be specially selected by screening for low quiescent currents. Clock speeds of processors and other logic circuits should be reduced to the minimum required for computational throughput as power consumption increases with frequency. Supply voltages should also be kept at minimal values consistent with reliable operation because power dissipation in charging internal capacitance's during switching is proportional to the square of the voltage. Where possible, supply voltages should be approximately the same throughout the circuit to prevent current flowing through input protection circuits. Logic inputs should not be left floating and circuits should be arranged so that power consumption is minimised in the most usual logic output state. Slow logic transitions are undesirable because they can result in relatively large class-A currents flowing. Resistors may be incorporated in the power supply to individual devices in order to minimise current in the event of failure.

In some control applications, devices that switch between on and off states are preferred to those that allow analog (e.g. linear) control because less power is dissipated in low resistance on states and low current off states. Where linear components are used (e.g. certain types of voltage regulators) then types with low quiescent currents should be selected. In some circuit configurations it is preferable to use appropriate reactive components (i.e. inductors and capacitors) to reduce power dissipation in resistive components.

Suitably, the system additionally comprises a visual display unit for display of data from the electronic data management system to the user. The display may for example, comprise a screen such as an LED or LCD screen. More preferably the visual display unit is associable with the body of the medicament dispenser.

Suitably, the medicament dispenser additionally comprises a datalink for linking to a local data store to enable communication of data between the local data store and the electronic data management system. The datastore may also comprise data management, data analysis and data communication capability.

The datastore may itself form part of a portable device (e.g. a handheld device) or it may be sized and shaped to be accommodated within the patient's home. The datastore may also comprise a physical storage area for storage of replacement cassettes. The datastore may further comprise a system for refilling medicament from a reservoir of medicament product stored therewithin. The datastore may further comprise an electrical recharging system for recharging any electrical energy store on the medicament dispenser, particularly a battery recharging system.

The datalink may for example enable linking with a docking station, a personal computer, a network computer system or a set-top box by any suitable method including a hard-wired link, an infrared link or any other suitable wireless communications link.

Suitably, the medicament dispenser additionally comprises an actuation detector for detecting actuation of the dispensing mechanism wherein said actuation detector transmits actuation data to the electronic data management system.

The medicament dispenser may additionally comprise a safety mechanism to prevent unintended multiple actuations of the dispensing mechanism. The patient is thereby protected from inadvertently receiving multiple doses of medicament in a situation where they take a number of short rapid breaths. More preferably, the safety mechanism imposes a time delay between successive actuations of the release means. The time delay is typically of the order of from three to thirty seconds.

Suitably, the medicament dispenser additionally comprises a release detector for detecting release of medicament from the cassette, wherein said release detector transmits release data to the electronic data management system.

Suitably, the medicament dispenser additionally comprises a shake detector for detecting shaking of the medicament container (e.g. prior to actuation of the dispensing mechanism), wherein said shake detector transmits shake data to the electronic data management system.

Suitably, any actuation detector, release detector, or shake detector comprises a sensor for detecting any suitable parameter such as movement. Any suitable sensors are envisaged including the use of optical sensors. The release detector may sense any parameter affected by release of the medicament such as pressure, temperature, sound, moisture, carbon dioxide concentration and oxygen concentration.

Suitably, the medicament dispenser additionally comprises a breath trigger for triggering the dispensing mechanism, said breath trigger being actuable in response to a trigger signal from the electronic data management system. Preferably, the electronic data management system includes a predictive algorithm or look-up table for deriving from the breath data when to transmit the trigger signal. For example, a real-time analysis of the patient breath waveform may be made and the trigger point derived by reference to that analysed waveform.

Suitably, the electronic data management system includes a predictive algorithm or look-up table for calculating the optimum amount of medicament to dispense.

Suitably, the memory on the electronic data management system includes a dose memory for storing dosage data and reference is made to the dose memory in calculating the optimum amount of medicament to dispense.

Suitably, the medicament dispenser additionally comprises a selector for selecting the amount of medicament to dispense from said dispensing mechanism. In one aspect, the selector is manually operable. In another aspect, the selector is operable in response to a signal from the transmitter on the electronic data management system.

Suitably, the medicament dispenser comprises in association with a body or housing thereof, a first transceiver for transmitting and receiving data and in association with the medicament container, a second transceiver for transmitting and receiving data, wherein data is transferable in two-way fashion from the first transceiver to the second transceiver. The data is preferably in digital form and suitable for transfer by electronic or optical means. A medicament dispenser of this general type is described in pending UK Patent Application No. 0020538.5.

One advantage of embodiments of this type is the ability to store many types of information in different parts of the memory structure of the transceivers. The information is furthermore stored in a form that is readily and accurately transferable. The information could for example, include manufacturing and distribution compliance information written to the memory at various points in the manufacturing or distribution process, thereby providing a detailed and readily accessible product history of the dispenser. Such product history information may, for example, be referred to in the event of a product recall. The compliance information could, for example, include date and time stamps. The information could also include a unique serial number stored in encrypted form or in a password protectable part of the memory that uniquely identifies the product and therefore may assist in the detection and prevention of counterfeiting. The information could also include basic product information such as the nature of the medicament and dosing information, customer information such as the name of the intended customer, and distribution information such as the intended product destination.

On loading or reloading the medicament dispenser with a cassette the second transceiver may, for example, read the unique serial number, batch code and expiry date of the medicament and any other information on the second transceiver. In this way the nature and concentration of the medicament, together with the number of doses used or remaining within the cassette, may be determined. This information can be displayed to the patient on a visual display unit. Other information, such as the number of times the medicament dispenser has been reloaded with a cassette, may also be displayed.

Similarly, should the cassette be removed from the holder before the supply of medicament is exhausted, the same data can be read from the second transceiver and the number of doses remaining or used determined. Other information, such as the date and time of administration of the drug, or environmental exposure data such as the minimum/maximum temperatures or levels of humidity the cassette has been exposed to, may also be read and displayed to the user.

In the event that the supply of medicament within the container becomes exhausted, or that the shelf life of the medicament has expired, or that the first transceiver does not recognise the batch code on the second transceiver, activation of the dispenser may be prevented to safeguard the user. Activation may also be prevented if the medicament has been exposed to extreme environmental conditions for periods outwith the manufacturer's guidelines.

Data may be transferred to and from any transceiver during the period of use of the medicament dispenser by the patient. For example, the medicament dispenser may include an electronic data management system having various sensors associated therewith. Any data collected by the sensors or from any data collection system associated with the electronic data management system including a clock or other date/time recorder is transferable.

Data may be transferred each time the patient uses the device. Or alternatively, data may be stored in a database memory of the electronic data management system and periodically downloaded to any transceiver. In either case, a history of the usage of the device may be built up in the memory of a transceiver.

In one embodiment herein, a history of the usage of the medicament dispenser is transferred to the second transceiver. When the blister strip in the cassette is exhausted it is exchanged by the patient for a new refill cassette. At the point of exchange, which will typically occur at the pharmacy, data may be transferred from the exhausted cassette to the refill and vice-versa. Additionally, usage history data may be read from the refill and transferred to a healthcare data management system for example comprising a network computer system under the control of a healthcare data manager.

Methods are envisaged herein whereby the patient is given some sort of reward for returning the refill and making available the data comprised within the second transceiver. Methods are also envisaged herein whereby the healthcare data manager is charged for either receipt of the data from the second transceiver or for its use for commercial purposes. Any rewards or charging may be arranged electronically. The methods may be enabled by distributed or web-based computer network systems in which any collected data is accessible through a hub on the network. The hub may incorporate various security features to ensure patient confidentiality and to allow selective access to information collected dependent upon level of authorisation. The level of user authorisation may be allocated primarily to safeguard patient confidentiality. Beyond this the level of user authorisation may also be allocated on commercial terms with for example broader access to the database being authorised in return for larger commercial payments.

Suitably, the first and second transceiver each comprise an antenna or equivalent for transmitting or receiving data and connecting thereto a memory. The memory will typically comprise an integrated circuit chip. Either transceiver may be configured to have a memory structure that allows for large amounts of information to be stored thereon. The memory structure can be arranged such that parts of the memory are read-only, being programmed during/after manufacture, other parts are read/write and further parts are password protectable. Initial transfer of information (e.g. on manufacture or one dispensing) to or from any transceiver can be arranged to be readily achievable by the use of a reader which is remote from the medicament dispenser, thereby minimising the need for direct product handling. In further aspects, the reader can be arranged to simultaneously read or write to the memory of multiple transceivers on multiple medicament dispensers.

A suitable power source such as a battery, clockwork energy store, solar cell, fuel cell or kinetics-driven cell will be provided as required to any electronic component herein. The power source may be arranged to be rechargeable or reloadable.

Suitably, data is transferable in two-way fashion between the first and second transceiver without the need for direct physical contact therebetween. Preferably, data is transferable wirelessly between the first and second transceiver.

Suitably, the first transceiver is an active transceiver and the second transceiver is a passive transceiver. The term active is used to mean directly powered and the term passive is used to mean indirectly powered.

Suitably, the second transceiver comprises a label or tag comprising an antenna for transmitting or receiving energy; and an integrated circuit chip connecting with said antenna, and the first transceiver comprises a reader for said label or tag. In this case the label or tag is a passive transceiver and the reader is an active transceiver. Preferably, the reader will not need to be in direct contact with the tag or label to enable the tag or label to be read.

The tag may be used in combination and/or integrated with other traditional product labelling methods including visual text, machine-readable text, bar codes and dot codes.

Suitably, the integrated circuit chip has a read only memory area, a write only memory area, a read/write memory area or combinations thereof.

Suitably, the integrated circuit chip has a one-time programmable memory area. More preferably, the one-time programmable memory area contains a unique serial number.

Suitably, the integrated circuit chip has a preset memory area containing a factory preset, non-changeable, unique data item. The preset memory item is most preferably in encrypted form.

Suitably, the integrated circuit chip has plural memory areas thereon. Suitably, any memory area is password protected.

Suitably, any memory area contains data in encrypted form. Electronic methods of checking identity, error detection and data transfer may also be employed.

In one aspect, the integrated circuit has plural memory areas thereon including a read only memory area containing a unique serial number, which may for example be embedded at the time of manufacture; a read/write memory area which can be made read only once information has been written thereto; and a password protected memory area containing data in encrypted form which data may be of anti-counterfeiting utility.

Suitably, the tag is on a carrier and the carrier is mountable on the body or holder of the medicament dispenser or on the cassette.

In one aspect, the carrier is a flexible label. In another aspect, the carrier is a rigid disc. In a further aspect, the carrier is a rectangular block. In a further aspect, the carrier is a collar ring suitable for mounting to the neck of an aerosol container. Other shapes of carrier are also envisaged.

Suitably, the carrier is mouldable or weldable to the cassette or housing. Suitably, the carrier encases the tag. More preferably, the carrier forms a hermetic seal for the tag.

In one aspect, the carrier comprises an insulating material such as a glass material or, a paper material or an organic polymeric material such as polypropylene. Alternatively, the carrier comprises a ferrite material.

The energy may be in any suitable form including ultrasonic, infrared, radio frequency, magnetic, optical and laser form. Any suitable channels may be used to channel the energy including fibre optic channels.

In one aspect, the second transceiver comprises a radio frequency identifier comprising an antenna for transmitting or receiving radio frequency energy; and an integrated circuit chip connecting with said antenna, and the first transceiver comprises a reader for said radio frequency identifier. In this case the radio frequency identifier is a passive transceiver and the reader is an active transceiver. An advantage of radio frequency identifier technology is that the reader need not be in direct contact with the radio frequency identifier tag or label to be read.

The radio frequency identifier can be any known radio frequency identifier. Such identifiers are sometimes known as radio frequency transponders or radio frequency identification (RFID) tags or labels. Suitable radio frequency identifiers include those sold by Phillips Semiconductors of the Netherlands under the trade marks Hitag and Icode, those sold by Amtech Systems Corporation of the United States of America under the trade mark Intellitag, and those sold by Texas Instruments of the United States of America under the trade mark Tagit.

Suitably, the antenna of the RFID tag is capable of transmitting or receiving radio frequency energy having a frequency of from 100 kHz to 2.5 GHz. Preferred operating frequencies are selected from 125 kHz, 13.56 MHz and 2.4 GHz.

In one aspect, the second transceiver comprises a magnetic label or tag comprising an antenna for transmitting or receiving magnetic field energy; and an integrated circuit chip connecting with said antenna, and the first transceiver comprises a reader for said magnetic label or tag. In this case the magnetic label or tag is a passive transceiver and the reader is an active transceiver.

A suitable magnetic label or tag comprises plural magnetic elements in mutual association whereby the magnetic elements move relative to each other in response to an interrogating magnetic field. A magnetic label or tag of this type is described in U.S. Pat. No. 4,940,966. Another suitable magnetic label or tag comprises a magneto restrictive element which is readable by application of an interrogating alternating magnetic field in the presence of a magnetic bias field which results in resonance of the magneto restrictive elements at different predetermined frequencies. A magnetic label of this type is described in PCT Patent Application No. WO92/12402. Another suitable magnetic label or tag comprising plural discrete magnetically active regions in a linear array is described in PCT Patent Application No. WO96/31790. Suitable magnetic labels and tags include those making use of Programmable Magnetic Resonance (PMR) (trade name) technology.

In another aspect, the second transceiver comprises a microelectronic memory chip and the first transceiver comprises a reader for said microelectronic memory chip. The microelectronic memory chip may comprise an Electrically Erasable Programmable Read Only Memory (EEPROM) chip or a SIM card-type memory chip. In this case the microelectronic memory chip is a passive transceiver and the reader is an active transceiver.

Any transceiver herein, particularly a passive transceiver may be mounted on or encased within any suitable inert carrier. The carrier may comprise a flexible sheet that may in embodiments be capable of receiving printed text thereon.

In one aspect, the first transceiver is integral with the body such that a single unit is comprised. The first transceiver may for example be encased within or moulded to the body.

In another aspect, the first transceiver forms part of a base unit that is reversibly associable with the body. The base unit may for example, form a module receivable by the body such as a snap-in module.

Suitably, the medicament dispenser additionally comprises a communicator for wireless communication with a network computer system to enable transfer of data between the network computer system and the electronic data management system. Dispensers employing such communicators are described in pending PCT Applications No.s PCT/EP00/09291 (PG3786), PCT/EP00/09293 (PG4029) and PCT/EP00/09292 (PG4159). Preferably, the communicator enables two-way transfer of data between the network computer system and the electronic data management system.

Suitably, the data is communicable between the network computer system and the electronic data management system in encrypted form. All suitable methods of encryption or partial encryption are envisaged. Password protection may also be employed. Suitably, the communicator employs radio frequency or optical signals.

In one aspect, the communicator communicates via a gateway to the network computer system. In another aspect, the communicator includes a network server (e.g. a web server) such that it may directly communicate with the network.

In a further aspect, the communicator communicates with the gateway via a second communications device. Preferably, the second communications device is a telecommunications device, more preferably a cellular phone or pager. Preferably, the communicator communicates with the second communications device using spread spectrum radio frequency signals. A suitable spread spectrum protocol is the Bluetooth (trade mark) standard that employs rapid (e.g. 1600 times a second) hopping between plural frequencies (e.g. 79 different frequencies). The protocol may further employ multiple sending of data bits (e.g. sending in triplicate) to reduce interference.

In one aspect, the network computer system comprises a public access network computer system. The Internet is one suitable example of a public access network computer system, wherein the point of access thereto can be any suitable entry point including an entry point managed by an Internet service provider. The public access network computer system may also form part of a telecommunications system, which may itself be either a traditional copper wire system, a cellular system or an optical network.

In another aspect, the network computer system comprises a private access network computer system. The private access network system may for example, comprise an Intranet or Extranet that may for example, be maintained by a health service provider or medicament manufacturer. The network may for example include password protection; a firewall; and suitable encryption means.

Preferably, the communicator enables communication with a user-specific network address in the network computer system.

The user-specific network address may be selected from the group consisting of a web-site address, an e-mail address and a file transfer protocol address. Preferably, the user-specific network address is accessible to a remote information source such that information from said remote information source can be made available thereto. More preferably, information from the user-specific network address can be made available to the remote information source.

In one aspect, the remote information source is a medicament prescriber, for example a doctor's practice. Information transferred from the medicament prescriber may thus, comprise changes to prescription details, automatic prescription updates or training information. Information transferred to the medicament prescriber may comprise compliance information, that is to say information relating to the patient's compliance with a set prescribing programme. Patient performance information relating for example, to patient-collected diagnostic data may also be transferred to the medicament prescriber. Where the dispenser is an inhaler for dispensing medicament for the relief of respiratory disorders examples of such diagnostic data would include breath cycle data or peak flow data.

In another aspect, the remote information source is a pharmacy. Information transferred from the pharmacy may thus, comprise information relating to the medicament product. Information sent to the pharmacy may thus include prescription requests which have been remotely pre-authorised by the medicament prescriber.

In a further aspect, the remote information source is an emergency assistance provider, for example a hospital accident and emergency service or an emergency helpline or switchboard. The information may thus, comprise a distress or emergency assist signal which requests emergency assistance.

In a further aspect, the remote information source is a manufacturer of medicament or medicament delivery systems. Information transferred to the system may thus, comprise product update information. The system may also be configured to feed information back to the manufacturer relating to system performance.

In a further aspect, the remote information source is a research establishment. In a clinical trial situation, information may thus be transferred relating to the trial protocol and information relating to patient compliance fed back to the research establishment.

In a further aspect, the remote information source is an environmental monitoring station. Information relating to weather, pollen counts and pollution levels may thus be made accessible to the system.

Suitably, the medicament dispenser additionally comprises a geographic positioning system such as a global positioning system or a system which relies on the use of multiple communications signals and a triangulation algorithm.

The medicament may comprise a capsule, pellet or tablet. Alternatively, the medicament may be in powdered form. Preferably, when in powdered form the medicament comprises a drug. Preferably the drug is selected from the group consisting of albuterol, salmeterol, fluticasone propionate and beclomethasone dipropionate and salts or solvates thereof and any combination thereof. Preferably said combination comprises salmeterol xinafoate and fluticasone propionate.

Suitably, the powdered medicament additionally comprises an excipient. Suitably, said excipient is a sugar.

In yet another aspect, the invention provides a kit of parts comprising a cassette as described supra, a holder for a cassette and a body wherein the holder is shaped to fit within said body and may be movable relative to said body.

In a further aspect, the invention provides a body and holder for use in the medicament dispenser described supra.

In still a further aspect, the invention provides a cassette for use in the medicament dispenser described supra.

In yet another aspect, the invention provides the use of a medicament dispenser as described supra.

### BRIEF DESCRIPTION OF THE DRAWINGS

The invention will now be described with reference to the accompanying drawings in which:

FIG. 1 shows a perspective view of a medicament carrier for use in accordance with the present invention;

FIG. 2a shows a perspective view of a blister strip advancement mechanism for use in accordance with one aspect of the invention;

FIG. 2b a side view of the advancement mechanism of FIG. 1a located within the housing of a medicament dispenser;

FIG. 3 shows an exploded perspective view of a medicament dispenser in accordance with another aspect of the present invention; and

FIG. 4 shows an exploded perspective view of a second medicament dispenser in accordance with a further aspect of the invention.

### DETAILED DESCRIPTION OF THE DRAWINGS

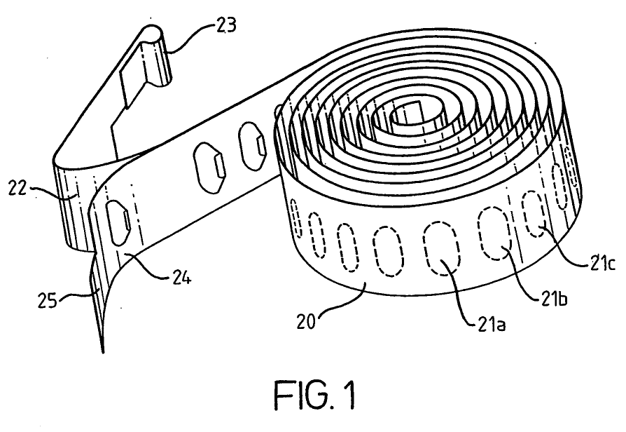

Referring now to the Figures, FIG. 1 shows a medicament carrier suitable for use in accord with the present invention. The medicament carrier comprises a flexible strip 20 defining a plurality of pockets 21a, 21b, 21c each of which contains a dose of medicament, which can be inhaled, in the form of powder.

The strip comprises a base sheet 24 in which blisters are formed to define the pockets 21a, 21b, 21c and a lid sheet 22 which is hermetically sealed to the base sheet 22 except in the region of the blisters in such a manner that the lid sheet 22 and the base sheet 24 can be peeled apart. The sheets 22, 24 are sealed to one another over their whole width except for the leading end portions 23, 25 where they are preferably not sealed to one another at all. The lid 22 and base 24 sheets are each preferably formed of a plastics/aluminium laminate and are preferably adhered to one another by heat sealing.

The strip 20 is shown as having elongate pockets 21a, 21b, 21c which run transversely with respect to the length of the strip 20. This is convenient in that it enables a large number of pockets 21a, 21b, 21c to be provided in a given strip 20 length. The strip 20 may, for example, be provided with sixty or one hundred pockets but it will be understood that the strip 20 may have any suitable number of pockets.

Embodiments are envisaged in which a medicament carrier strip 20 is supplied as a ‘refill’ to the medicament dispensers described herein (e.g. as illustrated in the other Figures). It is an advantage of the dispensers of the present invention that medicament carrier may be readily loaded in the internal mechanism thereof.

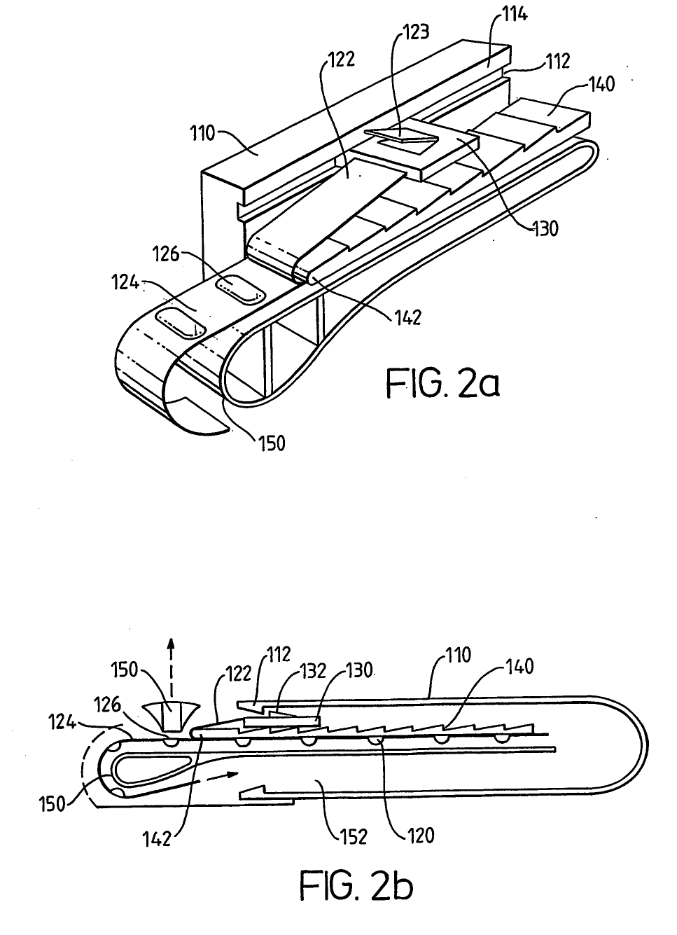

An internal mechanism according to one aspect of the invention is illustrated in FIG. 2a. That mechanism is illustrated within the housing 110 of a medicament dispenser in FIG. 2b.

In both FIGS. 2a and 2b, a first end 123 of the lid sheet 122 of a medicament carrier blister strip 120 is held under tension by anchor block 130. The anchor block 130 is itself mounted for lateral movement on a guide rail 112 formed within a wall 114 of the housing 110. That lateral movement of the anchor block 130 is however, subject to its relationship with linear index ratchet 140, which comprises plural ratchet teeth spaced along its length. It may also be seen that the forward edge of the index ratchet 140 is shaped to form an opening beak 142, which enables separation of the lid sheet 122 of the blister strip 120 from its base sheet 124 at an opening station. The base sheet 124 comprises blisters 126 for the containment of medicament spaced regularly there along. Exit passageway 150, which communicates with a mouthpiece (not shown), enables a patient to inhale medicament from an opened blister 126.

In use, the mechanism is first primed by indexed, lateral movement of the anchor block 130 along the guide rail 112 by one index ratchet 140 spacing. This indexing movement of the block 130 is achieved, as shown in FIG. 1b, by lateral movement of part of the housing 110 which causes dog tooth 112 located thereon to engage abutment 132 on the anchor block 130. In turn, movement of the block 130 results in the opening of a blister 126 of the strip 120 as lid sheet 122 is tensely pulled about the opening beak 142 thereby peeling it away from the base sheet 124. Whilst the base sheet 124 is essentially un-tensioned throughout, the opening peel action naturally results in its forward movement about crest 150 and towards receiving chamber 152 located within the housing 110.

In the embodiment of FIGS. 2a and 2b, the lateral movement of the anchor block is achieved by simple lateral movement of the housing 110 to give the engagement of abutment 132 and anchor block 130. In other embodiments, more sophisticated lateral actuation movements may be envisaged including those in which the housing 110 is movable along a rotary (e.g. screw drive) path and in which, the movement of the housing itself is subject to an index ratchet drive. In the latter embodiment, abutment 132 becomes ratchet teeth to form the ratchet index drive.

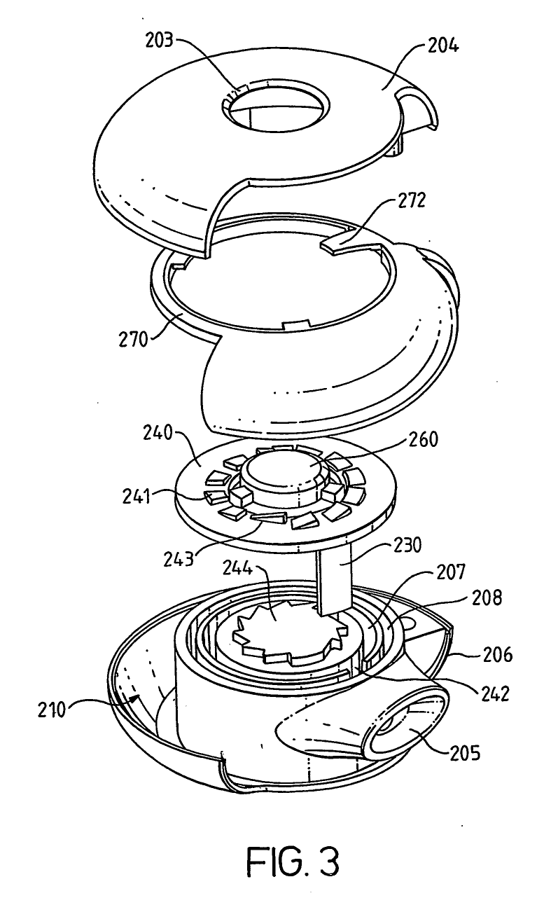

FIG. 3 shows a medicament dispenser herein suitable for use with a peelably separable elongate blister strip (as shown in FIG. 1). In the dispenser, an internal mechanism is located within a housing 210 having top 204 and bottom 206 covers. Whilst as shown in the drawing the top 204 and bottom 206 covers are shown as separate components embodiments are also envisaged in which these parts are hinged together and formed as a single moulded component.

The bottom cover 206 is provided with mouthpiece 205; first circular channel 207 shaped for receipt of a blister strip (not shown); and concentric therewith, a second circular channel 208 shaped for receipt of lid sheet anchor tab 230. The anchor tab 230 itself, protrudes from circular ratchet index 240, which is mounted for rotation and provided with plural ratchet teeth 241 arranged in radial series thereon. The ratchet index 240 is also provided with horizontally extending back ratchet 243 engagable with back ratchet gear 244 on the bottom cover to prevent reverse rotation thereof and a dose counter 260, which in use, is visible through a window 203 in the top cover 204. The ratchet index 240 is rotationally drivable by the engagement of ratchet drive tab 272 provided on mouthpiece cover 270 with the ratchet teeth 241 of the index 240. The mouthpiece cover 270 is also mounted for rotation within the housing 210.

In use, a blister strip is loaded into the dispenser such that the bulk of the (unopened) strip is held within the first circular channel 207 of the bottom cover 206, but a leading edge of the lid sheet of the blister strip is mounted for tension on the lid sheet anchor tab 230. To advance a dose, the mouthpiece cover 270 is rotated within the housing 210 causing ratchet drive tab 272 to index a ratchet tooth 241 of the ratchet index 240, which itself moves one indexed position forward. Rotation of the ratchet index 240 in turn, causes the anchor tab 230 holding the lid sheet of the blister strip to be advanced by one index position. This results in peeling of the lid sheet of the blister strip from its base sheet to access a medicament dose at opening station 242, which is inhaled through the mouthpiece 205. (Whilst the details of the opening action are not visible in FIG. 3, it will be appreciated that an opening mechanism making use of an opening beak 142 as shown in FIGS. 2a and 2b would be suitable). After the dose has been dispensed, the mouthpiece cover 270 is rotated to the mouthpiece 205 covered position. Further doses are accessible by repeating the steps described above.

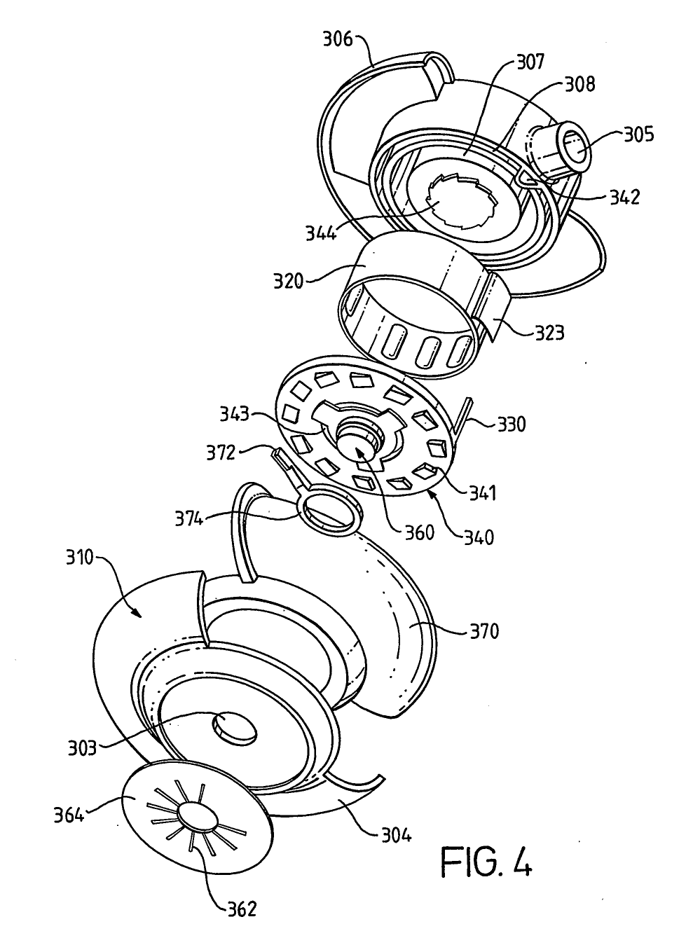

FIG. 4 shows a variation of the medicament dispenser of FIG. 3 herein, which is also suitable for use with a peelably separable elongate blister strip (as shown in FIG. 1). In the dispenser, an internal mechanism is located within a housing 310 having top 304 and bottom 306 snap-fit cover parts (e.g. formed of a transparent plastic material to make the inner workings visible to a patient). The bottom cover 306 is provided with mouthpiece 305; first circular channel 307 shaped for receipt of a blister strip 320 and concentric therewith, a second circular channel 308 shaped for receipt of lid sheet anchor tab 330. The anchor tab 330 is itself shaped to engage the leading end 323 of the lid sheet of blister strip 320. The tab 320 protrudes from circular ratchet index 340, which is mounted for rotation and provided with plural ratchet teeth 341 arranged in radial series thereon. The ratchet index 340 is also provided with a back ratchet 343 engagable with back ratchet gear 344 on the bottom cover 306 to prevent reverse rotation of the index 340. The ratchet index 340 is further provided with a dose indicator 360, which in use, is visible through a window 303 in the top cover 304. The dose indicator 360 includes an arrow, which references dose indicia 362 printed on dose indicator label 364 mounted around the window 303. The ratchet index 340 is rotationally drivable by the engagement of ratchet drive key 372 of ratchet drive piece 374 with the ratchet teeth 341 of the index 340. Whilst as illustrated, the ratchet drive key 372 has a single arm embodiments are also envisaged in which a dual-armed ratchet drive key is employed. The ratchet drive piece 374 is mounted for rotation within the housing 310, and is coupled to mouthpiece cover 370, which is also rotationally mounted.

In use, a blister strip 320 is loaded into the dispenser such that the bulk of the (unopened) strip 320 is held within the first circular channel 307 of the bottom cover 306, and the leading edge 323 of the lid sheet of the blister strip mounted for tension on the lid sheet anchor tab 330. To advance a dose, the mouthpiece cover 370 and, thereby the ratchet drive piece 374 is rotated within the housing 310 causing ratchet drive tab 372 to index a ratchet tooth 341 of the ratchet index 340, which moves one indexed position forward. Rotation of the ratchet index 340 in turn, causes the anchor tab 330 holding the lid sheet 323 of the blister strip 320 to be advanced by one index position. This results in peeling of the lid sheet 323 of the blister strip 320 from its base sheet to access a medicament dose at opening station 342, which is inhaled through the mouthpiece 305. Whilst the details of the opening action are not clearly visible in FIG. 4, it will be appreciated that the opening mechanism makes use of an opening beak 342 similar to that shown in FIGS. 2a and 2b. After the dose has been dispensed, the mouthpiece cover 370 is contra-rotated to again cover the mouthpiece 305. Further medicament doses are accessible by repeating the steps described above.

Whilst the medicament dispenser herein has been described in detail in relation to a dispenser capable of receiving a single elongate form medicament carrier embodiments are also envisaged in which plural such carriers are receivable for opening and delivery of the medicament contents thereof. In these embodiments, each medicament carrier may be associated with a separate driver and/or indexer and/or primer or alternatively, each medicament carrier may be associated with a common driver and/or indexer and/or primer.

It may be appreciated that any of the parts of the dispenser or cassette which contact the medicament suspension may be coated with materials such as fluoropolymer materials (e.g. PTFE or FEP) which reduce the tendency of medicament to adhere thereto. Any movable parts may also have coatings applied thereto which enhance their desired movement characteristics. Frictional coatings may therefore be applied to enhance frictional contact and lubricants (e.g. silicone oil) used to reduce frictional contact as necessary.

In one aspect, the medicament dispenser of the invention is suitable for dispensing medicament, particularly for the treatment of respiratory disorders such as asthma and chronic obstructive pulmonary disease (COPD), bronchitis and chest infections. The dispenser may also be suitable for dispensing medicament for use in other disease areas, for example the treatment of impotence.

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α, 9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R)-2-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino] propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Preferred medicaments are selected from albuterol, salmeterol, fluticasone propionate and beclomethasone dipropionate and salts or solvates thereof, e.g., the sulphate of albuterol and the xinafoate of salmeterol.

Medicaments can also be delivered in combinations. Preferred formulations containing combinations of active ingredients contain salbutamol (e.g., as the free base or the sulphate salt) or salmeterol (e.g., as the xinafoate salt) or formoterol (eg as the fumarate salt) in combination with an anti-inflammatory steroid such as a beclomethasone ester (e.g., the dipropionate) or a fluticasone ester (e.g., the propionate) or budesonide. A particularly preferred combination is a combination of fluticasone propionate and salmeterol, or a salt thereof (particularly the xinafoate salt). A further combination of particular interest is budesonide and formoterol (e.g. as the fumarate salt).

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (i.e. starch, cellulose and the like), lactose, glucose, mannitol, amino acids, and maltodextrins, and inorganic excipients such as calcium carbonate or sodium chloride. Lactose is a preferred excipient.

Particles of the powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The excipient may be included with the medicament via well known methods, such as by admixing, co-precipitating and the like. Blends of excipients and drugs are typically formulated to allow the precise metering and dispersion of the blend into doses. A standard blend, for example, contains 13000 micrograms lactose mixed with 50 micrograms drug, yielding an excipient to drug ratio of 260:1. Dosage blends with excipient to drug ratios of from 100:1 to 1:1 may be used. At very low ratios of excipient to drug, however, the drug dose reproducibility may become more variable.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. A medicament dispenser for use with a medicament carrier having a plurality of pockets for containing medicament wherein said pockets are spaced along the length of and defined between two peelable sheets secured to each other, said dispenser having an internal mechanism for accessing said medicament contained within said medicament carrier, said mechanism comprising,
a) an opening station for receiving a pocket of said medicament carrier;
b) associated with said opening station, a peeler for peelably opening a pocket, said peeler including a lid driver for drivably peeling a lid sheet from a base sheet of said pocket;
c) an outlet, positioned to be in communication with an opened pocket through which a user can access medicament from such an opened pocket; and
d) an indexer for indexing pockets of a medicament carrier in use with said medicament dispenser,
wherein said indexer comprises an index ratchet which is moveable between a locked position in which said ratchet locks said lid driver and a release position in which the ratchet releases the lid driver, and actuation of said medicament dispenser releases said index ratchet from the lid driver to enable drivable peeling of said lid sheet from said base sheet of the pocket at the opening station.

2. A medicament dispenser according to claim 1, wherein the index ratchet comprises a moveable ratchet arm.

3. A medicament dispenser according to claim 2, wherein said ratchet arm is rotationally mounted.

4. A medicament dispenser according to claim 2, wherein said ratchet arm is pivotally mounted.

5. A medicament dispenser according claim 1, further comprising a back ratchet to prevent reverse movement of the index ratchet.

6. A medicament dispenser according to claim 1, wherein in use, the lid driver applies tension to the lid sheet.

7. A medicament dispenser according to claim 1, wherein in use, the base sheet is untensioned.

8. A medicament dispenser according to claim 1, wherein the lid driver comprises a rotary drive mechanism.

9. A medicament dispenser according to claim 1, wherein the lid driver comprises a linear drive mechanism.

10. A medicament dispenser according to claim 1, additionally comprising a counter wherein actuation of said counter is coupled to the actuation of the medicament dispenser.

11. A medicament dispenser according to claim 10, wherein actuation of the counter is coupled to the release of the index ratchet.

12. A medicament dispenser according to claim 1, additionally comprising an indexing lever for actuating said dispenser.

13. A medicament dispenser according to claim 12, wherein said indexing lever comprises cam means for moving the index ratchet between locked and release positions.

14. A medicament dispenser according to claim 1, additionally comprising a moveable cover, wherein movement of said cover results in release of the index ratchet.

15. A medicament dispenser according to claim 1, wherein the lid driver comprises a mangle.

16. A medicament dispenser according to claim 1, wherein the lid driver comprises a roller.

17. A medicament dispenser according to claim 16, wherein said roller is composed of a polymeric rubber.

18. A medicament dispenser according to claim 1, wherein the index ratchet and/or the lid driver are operable by an electronic drive system.

19. A medicament dispenser according to claim 1, additionally comprising a first chamber to receive the medicament carrier when the base sheet and lid sheet are peelably sealed together and a second chamber to receive the base sheet after it has been separated from the lid sheet.

20. A medicament dispenser according to claim 19, wherein said first chamber and said second chamber are separated by a wall.

21. A medicament dispenser according to claim 20, wherein said wall is movable to adjust the size of the first and second chambers.

22. A medicament dispenser according to claim 21, wherein said wall is flexible to allow changes to the relative size of the first and second chambers.

23. A medicament dispenser according to claim 1, wherein the internal mechanism for accessing the said medicament contained within said medicament carrier is housed within a cassette.

24. A medicament dispenser according to claim 23 comprising,
a body;
a holder, shaped to fit within said body and movable relative to said body; and
receivable by said holder, said cassette containing said medicament carrier.

25. A medicament dispenser according to claim 24, wherein movement of said holder relative to said body results in movement of said cassette between a first position and a second position such that the cassette is reversibly removable from the holder when the cassette is in the second position.

26. A medicament dispenser according to claim 25, wherein the first position comprises a dispensing position.

27. A medicament dispenser according to claim 25, wherein the second position comprises a non-dispensing position.

28. A medicament dispenser according to claim 24, wherein the holder and body include attaching means to attach the holder to the body.

29. A medicament dispenser according to claim 28, wherein said attaching means comprise a pin and hole system.

30. A medicament dispenser according to claim 24, wherein the holder is pivotally movable relative to the body.

31. A medicament dispenser according to claim 24, wherein the holder is rotationally movable relative to the body.

32. A medicament dispenser according to claim 30, wherein the holder additionally comprises a stop to limit movement of the holder relative to the body to 180°.

33. A medicament dispenser according to claim 24, wherein the holder is slidably movable relative to the body.

34. A medicament dispenser according to claim 24, wherein the holder additionally comprises a catch to retain the cassette.

35. A medicament dispenser according to claim 34, wherein the catch is child resistant.

36. A medicament dispenser according to claim 24, wherein the cassette additionally comprises an indexing lever.

37. A medicament dispenser according to claim 24, wherein the cassette additionally comprises a mouthpiece.

38. A medicament dispenser according to claim 24, wherein the body covers the mouthpiece when the cassette is in the non-dispensing position.

39. A medicament dispenser according to claim 24, wherein the cassette additionally comprises a raised portion to fit against the holder.

40. A medicament dispenser according to claim 1, wherein the medicament is in powdered or solid form.

41. A medicament dispenser according to claim 40, wherein the medicament comprises a drug.

42. A medicament dispenser according to claim 41, wherein the drug is selected from the group consisting of albuterol, salmeterol, fluticasone propionate, beclomethasone dipropionate, salts thereof, solvates thereof, and any combination thereof.

43. A medicament dispenser according to claim 42, wherein said combination comprises salmeterol xinafoate and fluticasone propionate.

44. A medicament dispenser according to claim 41, wherein the medicament additionally comprises an excipient.

45. A medicament dispenser according to claim 44, wherein the excipient is a sugar.

46. A kit of parts comprising a cassette according to claim 24, a holder for said cassette and a body wherein the holder is shaped to fit within said body and is movable relative to said body.

47. A cassette for use in the medicament dispenser according to claim 24.

48. (canceled)
